(19) United States
(12) Patent Application Publication
(10) Pub. No.: US 2017/0333613 A1
(43) Pub. Date: Nov. 23, 2017

(54) Intravenous Sensing System

(71) Applicants: Ramin Berenji, El Centro, CA (US); Shahid Hussain, El Centro, CA (US)

(72) Inventors: Ramin Berenji, El Centro, CA (US); Shahid Hussain, El Centro, CA (US)

(21) Appl. No.: 15/158,736

(22) Filed: May 19, 2016

(51) Int. Cl. A61M 1/30; A61M 1/36; A61M 5/158

(52) U.S. Cl. CPC A61M 1/30; A61M 1/3659; A61M 2005/1586; A61M 2230/30; A61M 2230/005

(57) ABSTRACT

An intravenous sensing system includes an intravenous needle that may be inserted into an artery. Thus, the intravenous needle may receive blood from the artery. The intravenous needle may be fluidly coupled to a dialysis machine. Thus, the blood may be routed to the dialysis machine. A sensing unit is coupled to the needle. The sensing unit senses blood pressure inside the artery when the intravenous needle is inserted into the artery. The sensing unit is electrically coupled to the dialysis machine. Thus, the dialysis machine may monitor the blood pressure.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to sensing devices and more particularly pertains to a new sensing device for intravenously sensing blood pressure.

### BRIEF SUMMARY OF THE INVENTION

An embodiment of the disclosure meets the needs presented above by generally comprising an intravenous needle that may be inserted into an artery. Thus, the intravenous needle may receive blood from the artery. The intravenous needle may be fluidly coupled to a dialysis machine. Thus, the blood may be routed to the dialysis machine. A sensing unit is coupled to the needle. The sensing unit senses blood pressure inside the artery when the intravenous needle is inserted into the artery. The sensing unit is electrically coupled to the dialysis machine. Thus, the dialysis machine may monitor the blood pressure.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of an intravenous sensing system according to an embodiment of the disclosure.

FIG. 2 is a cross sectional view taken along line 2-2 of FIG. 1 of an embodiment of the disclosure.

FIG. 3 is a front view of an embodiment of the disclosure.

FIG. 4 is a perspective in-use view of an embodiment of the disclosure.

### DETAILED DESCRIPTION OF THE INVENTION

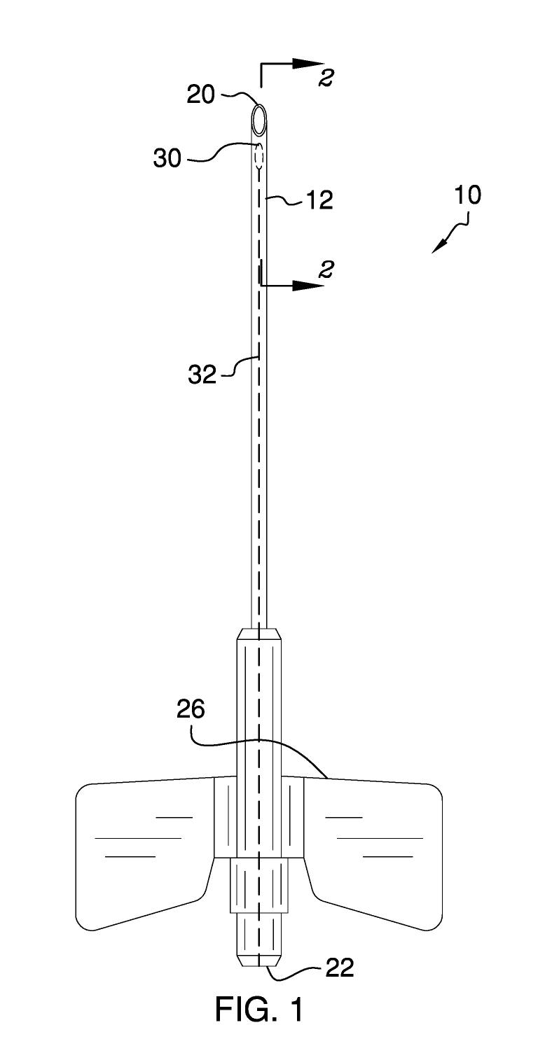

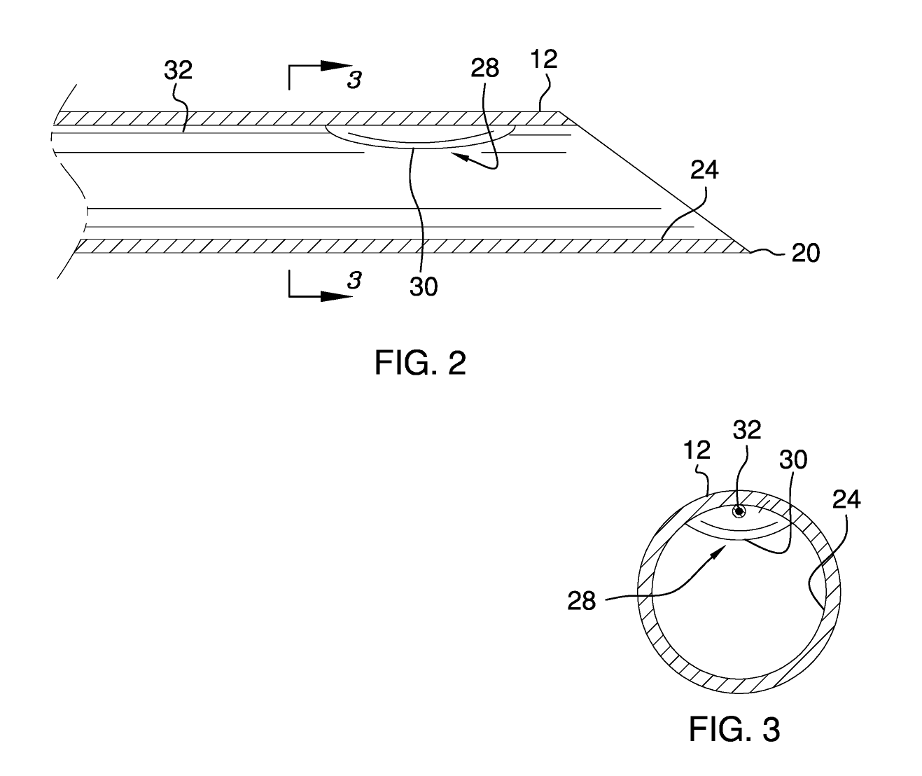

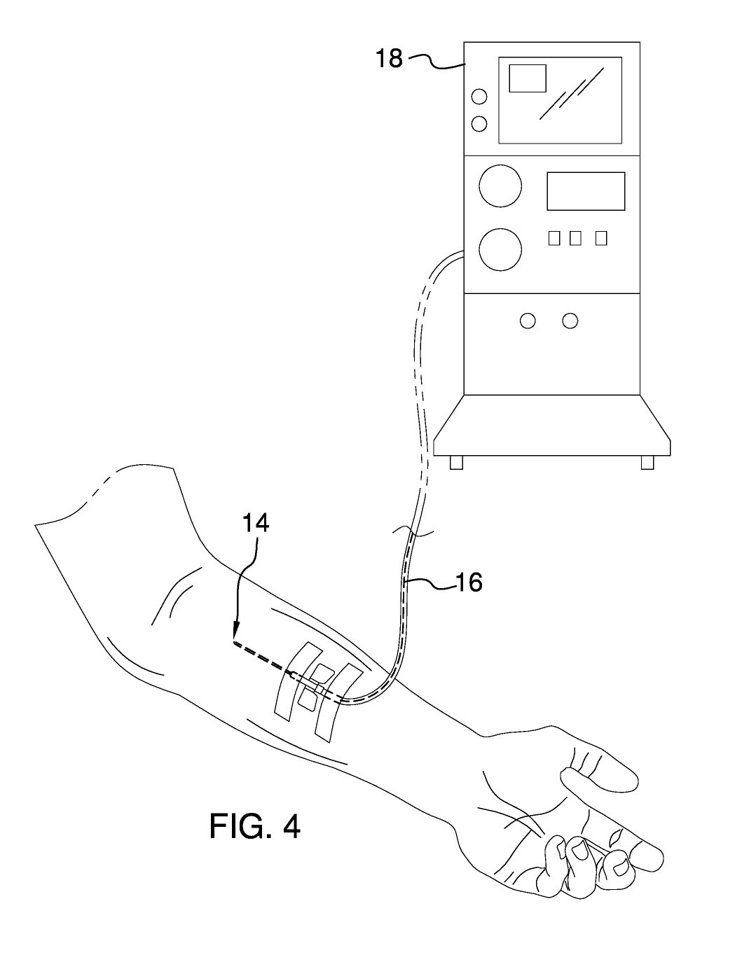

With reference now to the drawings, and in particular to FIGS. 1 through 4 thereof, a new sensing device embodying the principles and concepts of an embodiment of the disclosure and generally designated by the reference numeral 10 will be described.

As best illustrated in FIGS. 1 through 4, the intravenous sensing system 10 generally comprises an intravenous needle 12 that may be inserted into an artery 14. Thus, the intravenous needle 12 may receive blood 16 from the artery 14. The artery 14 may be a human artery. The intravenous needle 12 may be fluidly coupled to a dialysis machine 18. Thus, the blood 16 may be routed to the dialysis machine 18 for the purposes of dialysis.

The intravenous needle 12 has a first end 20 and a second end 22. Each of the first end 20 and the second end 22 is open and the intravenous needle 12 is substantially hollow. The intravenous needle 12 has an inner surface 24. The intravenous needle 12 may be a dialysis needle or the like.

A pair of wings 26 is attached to the intravenous needle 12. Each of the wings 26 is positioned adjacent to the second end 22. The first end 20 of the needle tapers to a point. Thus, the first end 20 may pierce a user's skin to enter the artery 14. Each of the wings 26 engages the user's skin thereby inhibiting the intravenous needle 12 from moving in the artery 14.

A sensing unit 28 is coupled to the needle 12. The sensing unit 28 senses blood pressure inside the artery 14 when the intravenous needle 12 is inserted into the artery 14. The sensing unit 28 is electrically coupled to the dialysis machine 18. Thus, the dialysis machine 18 may monitor the blood pressure in the artery 14.

The sensing unit 28 comprises a pressure sensor 30 that is coupled to the inner surface 24 of the intravenous needle 12. Thus, the pressure sensor 30 senses blood pressure when the intravenous needle 12 is inserted into the artery 14. The pressure sensor 30 is positioned closer to the first end 20 than the second end 22. The pressure sensor 30 comprises an electronic pressure sensor or the like. Moreover, the pressure sensor 30 comprises a medical grade pressure sensor.

A conductor 32 is electrically coupled to the pressure sensor 30. The conductor 32 extends along the inner surface 24 of the intravenous needle 12. The conductor 32 extends outwardly from the second end 22. Thus, the conductor 32 may be electrically coupled to the dialysis machine 18 thereby facilitating the dialysis machine 18 to monitor the blood pressure. The conductor 32 comprises a medical grade wire or the like.

In use, the intravenous needle 12 is manipulated in the convention of intravenous dialysis. The intravenous needle 12 is fluidly coupled to the dialysis machine 18 through any conventional medical means. The conductor 32 is electrically coupled to the dialysis machine. The pressure sensor 30 continually senses blood pressure in the artery 14 and the blood pressure is communicated to the dialysis machine 18. Thus, the dialysis machine 18 accurately monitors the blood pressure during dialysis.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous sensing system being configured to sense blood pressure inside an artery, said system comprising:
an intravenous needle being configured to be inserted into an artery thereby facilitating said intravenous needle to receive blood from the artery, said intravenous needle being configured to be fluidly coupled to a dialysis machine thereby facilitating the blood to be routed to the dialysis machine; and
a sensing unit being coupled to said needle wherein said sensing unit is configured to sense blood pressure inside the artery when said intravenous needle is inserted into the artery, said sensing unit being configured to be electrically coupled to the dialysis machine thereby facilitating the dialysis machine to monitor the blood pressure.

2. The system according to claim 1, wherein said intravenous needle has a first end and a second end, each of said first end and said second end being open, said intravenous needle being substantially hollow, said intravenous needle having an inner surface.

3. The system according to claim 2, wherein said sensing unit comprises a pressure sensor being coupled to said inner surface of said intravenous needle wherein said pressure sensor is configured to sense blood pressure when said intravenous needle is inserted into the artery, said pressure sensor being positioned closer to said first end than said second end.

4. The system according to claim 2, further comprising:
a pressure sensor being configured to sense blood pressure; and
a conductor being electrically coupled to said pressure sensor, said conductor extending along said inner surface of said intravenous needle, said conductor extending outwardly from said second end wherein said conductor is configured to be electrically coupled to the dialysis machine thereby facilitating the dialysis machine to monitor the blood pressure.

5. An intravenous sensing system being configured to sense blood pressure inside an artery, said system comprising:
an intravenous needle being configured to be inserted into an artery thereby facilitating said intravenous needle to receive blood from the artery, said intravenous needle being configured to be fluidly coupled to a dialysis machine thereby facilitating the blood to be routed to the dialysis machine, said intravenous needle having a first end and a second end, each of said first end and said second end being open, said intravenous needle being substantially hollow, said intravenous needle having an inner surface; and
a sensing unit being coupled to said needle wherein said sensing unit is configured to sense blood pressure inside the artery when said intravenous needle is inserted into the artery, said sensing unit being configured to be electrically coupled to the dialysis machine thereby facilitating the dialysis machine to monitor the blood pressure, said sensing unit comprising:
a pressure sensor being coupled to said inner surface of said intravenous needle wherein said pressure sensor is configured to sense blood pressure when said intravenous needle is inserted into the artery, said pressure sensor being positioned closer to said first end than said second end, and
a conductor being electrically coupled to said pressure sensor, said conductor extending along said inner surface of said intravenous needle, said conductor extending outwardly from said second end wherein said conductor is configured to be electrically coupled to the dialysis machine thereby facilitating the dialysis machine to monitor the blood pressure.
